(19) United States
(12) Patent Application Publication
(10) Pub. No.: US 2026/0315157 A1
(43) Pub. Date: Oct. 15, 2026

(54) 3D MARKER ELEMENT, MEDICAL PRODUCT, TRACKING SYSTEM AND POSITION DETECTION METHOD FOR SPATIAL TRACKING

(71) Applicant: B. Braun New Ventures GmbH, Freiburg im Breisgau (DE)

(72) Inventor: Jean Stawiaski, Freiburg (DE)

(21) Appl. No.: 19/482,954

(22) PCT Filed: May 16, 2024
(86) PCT No.: PCT/EP2024/063559

§ 371 Date: Nov. 10, 2025

(30) Foreign Application Priority Data
May 17, 2023 (DE) 10 2023 113 085.4

(51) Int. Cl. A61B 34/20; A61B 90/00; A61B 90/92; A61B 90/94; G06T 7/73

(52) U.S. Cl. CPC A61B 34/20; A61B 90/92; A61B 90/94; G06T 7/74; A61B 2034/2055; A61B 2034/2065; A61B 2090/371; A61B 2090/3937; A61B 2217/005; G06T 2207/30004; G06T 2207/30204

(57) ABSTRACT

A surgical 3D marker element has a surface, on or in which a two-dimensional optical pattern is permanently incorporated or applied, for spatial position detection of a medical product. The optical pattern is in the form of a matrix with M rows and N columns with corresponding pattern elements as pattern fields. The optical pattern has at least four pattern elements, of which at least two pattern elements are designed differently to provide an optical reference for position detection of the 3D marker element via an optical tracking system. The 3D marker element can be part of or used with a medical product, a tracking system, a position detection method, a computer-readable storage medium and a computer program.

### CROSS-REFERENCE TO RELATED APPLICATIONS

This application is the United States national stage entry of International Application No. PCT/EP2024/063559, filed on May 16, 2024, and claims priority to German Application No. 10 2023 113 085.4, filed on May 17, 2023.

### FIELD

The present disclosure relates to a surgical (optical) 3D marker element having a surface, in particular a sterilizable or sterilized surface for use in surgery, with a two-dimensional optical pattern which may be used for spatial pose recognition of a medical product. In addition, the present disclosure relates to a medical product, a tracking system, a pose recognition method as well as a computer-readable storage medium and computer program.

### BACKGROUND

Spatial tracking and estimation of a pose of a (medical) object, such as an instrument or an apparatus (such as with a handpiece) based on a 2D image/2 D picture requires a precise appearance model of the object. Usually, central, characteristic points or key points of the object are recognized and compared with a (stored) basic model/template. Knowledge of a 2D to 3D correspondence or a 3D to 3D correspondence makes it possible to calculate or estimate the pose of the object, i.e. its position and orientation. Estimating the Euclidean pose of an object based on its image also requires the image to be captured by a calibrated camera.

There are currently various classic solutions for the spatial tracking of surgical instruments, which are based on the following tracking methods and systems:
infrared markers are attached around a handle of the instrument, which are detected by a camera; in particular, at least four infrared markers are attached in the form of spheres (rigid bodies),
planar, optically visible markers or optical patterns, such as a QR code, are attached to or around a handle of an instrument, which are detected by a camera,
colored or elongated markers are attached to a tip of the instrument, which are used for tracking.

However, surgical instruments usually do not have enough distinguishing features on their surface so that classical computer vision algorithms cannot be used to estimate their pose. Their appearance also poses a challenge. For example, metallic surfaces are often reflective and smoothly configured, so that no or hardly any visual features can be extracted from an image (too little contrast, etc.).

As a result, it is often not possible to ensure precise determination of a pose of an instrument without other geometric objects such as rigid bodies or something similar on the instrument, which jeopardizes patient safety during surgery. In addition, instruments currently have to be elaborately extended and SUMMARY It is therefore the object of the present disclosure to avoid or at least reduce the disadvantages of the prior art and in particular to provide a surgical 3D marker element, a medical product, a tracking system, a pose recognition method as well as a computer-readable storage medium as well as a computer program with which precise and effective spatial pose recognition can be performed via optical detection. A further partial object can be seen in particular in the cost-effective manufacture and adaptation of medical products and tracking systems. Furthermore, another partial object in particular can be seen in the optical (encrypted) provision of further data in addition to pose recognition.

A basic idea thus provides integrating a special optical pattern directly on or in a surface, which is specially adapted and optically detectable. On the basis of a 2D image or a 3D image of the 3D marker element with the defined optical pattern, pose recognition may then be performed via a tracking system, in particular a navigation system with a camera.

In other words, such a marker design or 3D marker element is proposed which may be printed or engraved on or in surfaces, in particular also on medical products such as surgical instruments. This defined 3D marker element then enables the calculation of the pose (if appropriate also that of an instrument) with six degrees of freedom (6-DOF—Degree of Freedom). The 3D marker element may be provided on portions or parts of a medical product, in particular may be printed or engraved, in particular lasered, on portions of an instrument, for example on a distal portion of a suction cannula or a navigation pointer/navigation indicator. In this way, a pose detection element may be created simply and effectively as well as optimized in terms of installation space (no further elements need to be added) and may be attached to a medical product, for example, or integrated directly into it, such as engraved into a surface using a laser.

Specifically, a surgical 3D marker element with a surface, in particular a sterilizable surface, is proposed, wherein a two-dimensional optical pattern is permanently applied or incorporated on or in this surface, in particular is printed on or engraved (for example by laser engraving), for spatial pose recognition of a medical product. The optical 3D marker element on or in a visible (outer) surface thus provides a pose detection reference. According to the disclosure, the optical pattern (on or in the surface) is configured in the form of a matrix with M rows and N columns (i.e. a matrix M times N similar to a table) with corresponding (individual) pattern elements/modules as pattern fields of the matrix, and the optical pattern comprises at least four pattern elements (as pattern fields), of which at least two pattern elements are configured differently to each other in order to provide (as a minimum requirement) an optical reference for pose detection (position and orientation, i.e. six degrees of freedom—6DoF) of the 3D marker element via an (external) optical tracking system.

In a sense, a design guideline for the production of such a (visual) 3D marker element is described here, which may be used in particular for tracking surgical instruments. These, in particular rectilinear, 3D marker elements are, for example, mapped onto a cylindrical or flat surface, i.e. applied or introduced onto this surface. The optical pattern of the 3D marker element is divided into M times N (in particular equally sized) cells or pattern fields. The visual appearance of each pattern element is created in particular from a predefined dictionary of pattern-element types. The recognition of the optical pattern and of the visible pattern elements (i.e. of the cells or of the pattern fields filled by the pattern elements) may be performed using classical image processing algorithms based on machine learning or deep learning.

In particular with optical patterns larger than four pattern elements, it is advantageous that not all pattern elements need to be visible for pose determination. A minimum number of four keypoints have to be visible in order to determine a 6D position of the marker (3D position and 3D orientation). This optical pattern therefore does not have to be completely visible in order to be recognized, identified and spatially tracked.

In other words, it can also be said that for the 3D marker element, a special, in particular rectilinear, marker design is applied or introduced on a surface, in particular on or in a cylindrical part of an instrument or a flat surface, in particular is printed on or engraved. The marker design is prepared in such a way that visual cell features or the visual differences of the pattern elements are used to uniquely identify each cell or pattern field.

In particular, the optical patterns of two different 3D marker elements may be configured differently for different instrument models to ensure differentiation of the instrument type. Thus, a specific instrument model can be identified by the design of the optical pattern of the 3D marker element.

The term ‘position’ refers to a geometric position in three-dimensional space, which in particular is specified using coordinates of a Cartesian coordinate system. In particular, the position can be specified by the three coordinates X, Y and Z.

The term ‘orientation’ in turn indicates an alignment (for example at the position) in space. It can also be said that the orientation indicates an alignment with direction or rotation in three-dimensional space. In particular, the orientation may be specified using three angles.

The term ‘pose’ includes both a position and an orientation. In particular, the pose can be indicated via six coordinates, three position coordinates X, Y and Z and three angular coordinates for the orientation.

Advantageous embodiments are explained in particular below.

According to an embodiment, the surface may be curved, preferably may be a portion of a cylindrical lateral surface. This means that the surface is not a planar/level surface or does not have to be a planar surface.

According to an embodiment, the optical pattern may be configured in the form of a rectangular matrix with corresponding rectangular pattern fields each having the same outline shape, in particular with at least eight rows and/or at least five columns. Such a standardized optical pattern can be implemented easily and efficiently and reflects standardized geometric relations. The pattern elements, which may be taken from a dictionary, are then inserted into the individual pattern fields of the matrix. In particular, such a configuration can be easily applied to or inserted into a surface and reliable spatial tracking may be realized.

In particular, the surface (with the optical pattern) may be configured to be level/planar (i.e. flat) or may be configured to be curved, such as a portion of a cylindrical surface.

According to an embodiment, the matrix may have a non-square configuration. This means that the matrix in particular cannot be a QR code.

According to an embodiment, the different pattern elements may be arranged irregularly over a portion of the optical pattern, over a majority of the optical pattern or over the entire optical pattern, in particular not in rows or columns with exclusively identical pattern elements. This means that the matrix cannot be a barcode in particular.

According to an embodiment, the matrix may have a larger number of rows than columns. Preferably, the matrix may have at least 1.2 times, or 1.5 times, or 2 times as many and/or at most 5 times, or 4 times, or 3 times as many rows as columns. Such a configuration is advantageous in particular for attachment to shaft instruments configured in a rather elongated manner, in particular circular-cylindrical shaft instruments.

In particular, the optical pattern may have exactly two or more than two, preferably exactly three, or exactly four, or exactly five, different pattern elements (as pattern dictionary), which differ from each other at least in one color or color perception (for example by different contrasts), and furthermore preferably by a circle, or ellipse, or dot centered in the pattern field or a rectangle or rectangular outline centered in the pattern field, in particular each in a further color. The term ‘color’ refers here to black and white. If a single 3D marker element is created, it is first determined how many pattern elements it should have. If, for example, only binary pattern elements are used, a distinction can be made easily and reliably, although the optical pattern has to of course be made larger by the rows and columns for ‘encoding’ additional data such as an instrument type. In particular, two high-contrast colors are suitable for a differentiation or an engraving in a surface, there are the two ‘cases’ that a pattern field has not been engraved and thus forms a first pattern element, whereas a second pattern field has been engraved and thus forms a second pattern field with a different color (or color perception) (for example, because it is matte and thus appears different from the smooth surface or because the light is only partially reflected and thus appears darker). The number of pattern elements may be increased via the other geometric elements, such as a circle or dot (e.g. in the case of laser engraving), so that the optical pattern can still be created easily and cost-effectively in the surface.

According to a further embodiment, the optical pattern may have exactly two different pattern elements as binary pattern elements (one may also say form an entry of a binary pattern group in the dictionary), with the following preferred embodiments: a first pattern element of a first color, in particular white pattern field (in particular not engraved), and a second pattern element of second color, in particular as black, (in particular engraved) pattern field (Here it is crucial that the pattern fields in particular differ in contrast in order to be distinguished by an image of a camera); or a first pattern element as a light, in particular white, pattern field with a central dark, in particular black, dot or ellipse, and a second pattern element as a dark, in particular black, pattern field with a central light, in particular white, dot ellipse; or a first pattern element as a light, in particular white, pattern field with a central dark, in particular black, rectangle, in particular a square, and a second pattern element as a dark, in particular black, pattern field. In the present case, a binary cell type or pattern-element type is formed from two different visual appearances, which form the pattern elements of the optical pattern.

Alternatively, the optical pattern may preferably have exactly three different pattern elements as ternary pattern elements, with the following preferred embodiments: a first pattern element as a light, in particular white, pattern field, a second pattern element as a dark, in particular black, pattern field, and a third pattern element in a further (different) color, in particular red; or a first pattern element as a light, in particular white, pattern field with a central dark, in particular black, dot or ellipse, a second pattern element as a dark, in particular black, pattern field with a central light, in particular white, dot or ellipse, and a third pattern element as a light, in particular white, pattern field; or a first pattern element as a light, in particular white, pattern field with a central dark, in particular black, rectangle, in particular square, a second pattern element as a dark, in particular black, pattern field with a central light, in particular white, rectangle, in particular square, and a third pattern element as a light, in particular white, pattern field with a central dark, in particular black, rectangle outline. A ternary cell type or pattern-element type is formed from three different visual appearances, so that even more information may be integrated into a matrix of the same size relative to the binary pattern elements (or binary dictionary entry).

Alternatively, the optical pattern may preferably have exactly four different pattern elements as quaternary pattern elements, with in particular: a first pattern element as a white pattern field, a second pattern element as a black pattern field, a third pattern element in a further first color, in particular red, and a fourth pattern element in a further, second color, in particular green, or a first pattern element as a white pattern field with a central black dot, a second pattern element as a black pattern field with a central white dot, a third pattern element as a white pattern field, and a fourth pattern element as a black pattern field. A quaternary cell type is formed from four different visual appearances.

According to one embodiment, the optical pattern may have at least three rows by three columns (3×3), so that a central pattern element is adjacent to (surrounded by) eight pattern elements, which together form a pattern word and can be uniquely identified. The marking is obtained by assigning each cell to a type of the predefined dictionary. A pattern element or a cell on a, in particular rectilinear, grid has eight direct neighbors. The concatenation of the cell type and the 8 direct neighboring cell types form the so-called cell word/pattern word. In particular, each cell in the matrix is connected to a different word. This special property makes it possible to develop efficient image processing algorithms that can identify each cell of a marker using only local picture information. In particular, each cell/pattern element of a marker may be uniquely identified by analyzing the neighboring cell types/pattern-element types.

Preferably, different pattern elements may be distinguished by color and/or center of a circle, and/or corners, and/or contours. In a sense, different dictionaries or a dictionary with different groups of entries with visual characteristics may be used to create a 3D marker element. Different 3D marker elements may also be created from the same dictionary (or group of entries, e.g. entry group of binary pattern elements), for example with binary pattern elements. This feature makes it possible to associate different 3D marker elements with different medical products, in particular instruments. It should be noted that for the problem of pose determination (determination of the positioning), all visual features available in the dictionary may be used, which in particular is one or a combination of the following features: center of a circle, corners, edges, contours.

According to one embodiment, a first portion of the optical pattern may be configured to be offset and/or inclined with respect to a second portion of the optical pattern such that the first portion and the second portion are not visible at the same time. Such a formation occurs in particular when the surface is not configured flat/planar/level.

According to an embodiment, the optical pattern may not contain any coded information. This means that the optical pattern is primarily used for purely visual recognition of the pose and/or in particular is not a barcode and/or is not a QR code.

With regard to a medical product, in particular a medical instrument or a medical apparatus, with or for optical pose recognition during an intervention on a patient, the object is solved in that the medical product has the surgical 3D marker element according to the present disclosure, in particular on a distal portion of the medical product, wherein preferably the 3D marker element is integrated directly into the medical product and the optical pattern is printed on or incorporated, for example engraved, in particular laser-engraved, into a surface of the medical product.

In one embodiment, the medical product may be a surgical instrument with a distal instrument tip, in particular a scalpel or a trocar or a navigation pointer, and in an area less than 50%, in particular less than 30%, particularly preferably less than 10% of a total length of a longitudinal axis away from the distal tip, in particular directly in the region of the instrument tip, the 3D marker element may be arranged to provide a spatial pose recognition of the instrument tip, in particular in a surgical scalpel as an instrument which has the 3D marker element on a flat surface of the cutting edge. Thus, if the optical marker element is arranged on a distal portion, for example on a pointer, its pose can be detected when it enters the field of view of a digital surgical microscope, which is used for tracking and corresponding navigation, and this pose may be integrated into the digital data accordingly. The special configuration allows simple and safe tracking.

According to a further embodiment, the medical product may have a cylindrical contour with a corresponding cylindrical surface on a distal portion, and the 3D marker element may be arranged partially circumferentially, in particular over the entire circumference, in particular may be engraved, wherein preferably the medical product is a medical suction device with a distal suction pipe/suction cannula, wherein the 3D marker element with the optical pattern is provided on the suction pipe, in particular the optical pattern is printed on or engraved into the surface of the suction pipe. In particular, a suction pipe may be used as a pointer to define characteristic points, for example. This form of the 3D marker element thus extends the functional possibilities of medical products.

In particular, the medical product may be a medical instrument with a special, in particular conical, geometry of a distal portion and an optical template pattern in the form of a rectangular matrix may be mapped adapted/deformed (distorted) to the special geometry, in particular the conical geometry, via a linear transformation matrix and may be present as an optical pattern adapted/projected in a deformed (distorted) manner on or in the surface. One could also say that the design of the template may be deformed or cut out in order to be mapped as an optical pattern on any surface. For example, if a digital template of the optical pattern is available as a rectangular matrix with corresponding pattern elements, this template may be adapted to a target surface via corresponding matrix transformation (linear algebra) and may be applied to or inserted into it. Similar to the analogy of an elastic rubber surface (known from a balloon), on which a homogeneously stretched rectangular matrix with pattern elements is initially printed, this may then be applied to an object and the optical pattern is distorted accordingly, but is still recognizable in its structure. In particular, special instrument geometries such as tweezers or conical instruments require special optical patterns that are not rectilinear. Such a design may be achieved in particular by masking or mapping a rectilinear marker design template onto the geometry of the surface, in particular that of the instrument to be tracked. Alternatively, a rectilinear marker design template may be deformed onto the instrument surface as an optical pattern by deforming the rectilinear cell grid. The marker may be applied or inserted on any type of surface, in particular by deforming and/or cutting out a rectilinear marker design.

With regard to a surgical tracking system, in particular a navigation system, for a surgical intervention on a patient, the objects are solved in that this surgical tracking system comprises the following system components: a surgical 3D marker element or a medical product according to the present disclosure (with the corresponding optical pattern), and a tracking unit/tracking device with at least one camera, preferably at least two cameras such as a stereo camera for a 3D image, which is adapted to optically detect the 3D marker element with the optical pattern and to determine a pose (with respect to the tracking unit) on the basis of the detected optical pattern and in particular to include it in a navigation system and to visually output a pose by a display device. The tracking system is specially adapted to use the optical pattern for pose recognition, in particular by providing the tracking system with definitions and relations to optical patterns, which it can process accordingly.

In particular, the surgical tracking system may further comprise a storage unit in which a dictionary of (groups of) pattern elements is stored, in particular binary pattern elements, and/or ternary pattern elements, and/or quaternary pattern elements, and a control unit may be adapted to read out the optical pattern on the basis of the dictionary and to determine a pose accordingly, wherein preferably already a part of the optical pattern is sufficient to determine the pose.

According to an embodiment, any portion of the optical pattern with at least four pattern elements may be usable for pose determination. This means that not the complete pattern, but only a portion with at least four pattern elements has to be visible in order to determine the pose.

According to an embodiment, the optical pattern may preferably be usable for pose determination purely optically, in particular without using information encoded in the optical pattern. This means that no additional information encoded in the optical pattern is required for unambiguous pose determination.

Preferably, a data set associated with the optical pattern may be stored in a storage unit, with in particular a transformation from the pose of the optical pattern to an operating point of a medical product, such as an instrument tip (e.g. a vector or an indication in the form of 1 cm in the direction of orientation (such as the longitudinal axis), 3 mm offset from the center of the longitudinal axis (such as with an angle) in order to detect the position of the operating point as well as an orientation of the distal portion, and a control unit may be adapted to determine the operating point as well as orientation of the distal portion on the basis of the pose recognition of the optical pattern as well as the stored transformation, and to output them visually in a view by the display device. This provides the surgeon with important information for his/her intervention.

With regard to a pose recognition method for a pose recognition of a 3D marker element with an optical pattern or of a medical product according to the present disclosure, the object is solved by the steps of: creating a 2D image by a camera of a tracking unit or a 3D image by at least two cameras of a tracking unit and detecting each visible pattern element of the optical pattern; identifying each pattern element by a control unit based on a dictionary with different pattern elements, in particular binary pattern elements, or ternary pattern elements, or quaternary pattern elements; determining a correspondence between a 2D position in the 2D image and a position of the template of the 3D marker element with the optical pattern or a correspondence between a 3D position in the 3D image and a position of the template of the 3D marker element with the optical pattern, performing an n-point perspective position determination (estimation) on the 2D image for a pose determination or a 3D-to-3D-point determination for a pose determination of the optical pattern. In other words, it may be said that the determination/calculation or at least estimation of the position of such a marker comprises: recognition of each cell/pattern element visible in an image; identification of each cell giving a correspondence between a 2D position on the image and a position on the theoretical 3D marker design; use of a perspective n-point position estimate based on the calculated correspondences. The pose/positioning determination may be extended to a 3D camera or stereo camera: recognition of each visible cell in the two stereo images; identification of each cell that gives a correspondence between a 2D position on the images and a position on the theoretical 3D marker design. The two positions on the stereo images may be triangulated, resulting in a 3D cell position; using 3D-to-3D point matching to estimate the pose of the 3D marker element.

With respect to a computer-readable storage medium or a computer program, the objects are solved by comprising instructions which, when executed by a computer, cause the computer to perform the steps of the pose recognition method according to the present disclosure.

Any disclosure in connection with the 3D marker element according to the present disclosure also applies to the medical product, the tracking system, and the pose recognition method according to the present disclosure, just as any disclosure in connection with the medical product or the tracking system, or the pose recognition method applies to the 3D marker element or the respective other preambles.

### BRIEF DESCRIPTION OF THE DRAWINGS

The present disclosure is explained in more detail below with reference to preferred embodiments with the aid of figures.

FIG. 1 shows a schematic front view of a surgical tracking system with a medical product with a 3D marker element, in each case according to a first preferred embodiment;

FIG. 2 shows a sketch of an as yet unfilled rectangular matrix with rectangular pattern fields for an optical pattern;

FIG. 3 shows an exemplary 3D marker element with an optical pattern, in which a partial area is shown enlarged for illustration purposes with a central pattern element and eight adjacent pattern elements;

FIGS. 4a to 4c show each examples of binary pattern elements, with the hatching representing a different color perception;

FIGS. 5a to 5c show each examples of ternary pattern elements;

FIGS. 6a and 6b show each examples of quaternary pattern elements;

FIG. 7 shows a schematic illustration of an image of a template of an optical pattern of binary type on a surface of a medical product;

FIG. 8 shows schematic illustration of an image of a template of an optical pattern of quaternary type on a surface of a medical product; and

FIG. 9 shows a flowchart of a pose recognition method according to a preferred embodiment.

The figures are schematic in nature and are only intended to aid understanding of the invention. Identical elements are marked with the same reference signs. The features of the various embodiments may be interchanged.

### DETAILED DESCRIPTION

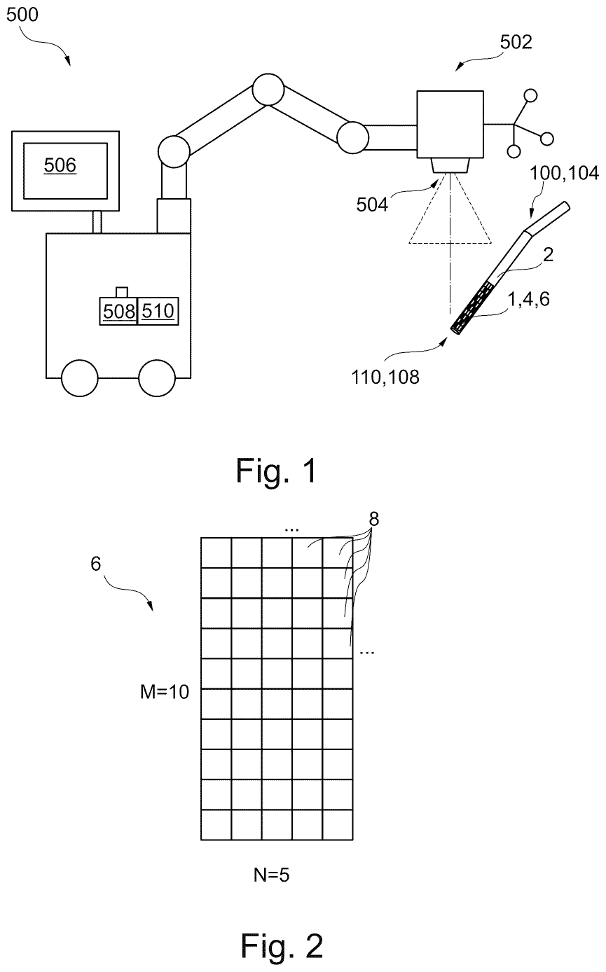

FIG. 1 shows a schematic front view of a surgical tracking system 500 in the form of a navigation system for tracking a surgical instrument, in particular during a surgical procedure on a patient (not shown) according to a preferred embodiment.

This tracking system 500 has a tracking unit 502 with a camera 504 in the form of a surgical microscope for spatial tracking. Furthermore, the tracking system 500 has a medical product 100 of a preferred embodiment in the form of a medical apparatus 104 of a suction device with a distal suction pipe 110, which is to be spatially tracked with respect to a pose, i.e. a position and an orientation, using the camera 504. This suction pipe 110 has a surgical 3D marker element 1 on its distal portion 108 according to a preferred embodiment. For example, a statement about the position and/or orientation of the medical product 100 in relation to the surgical microscope is sufficient in order to adjust the pose of the surgical microscope. Optionally, the surgical tracking system 500 has an additional external navigation system with a marker on the surgical microscope, in particular on the microscope head, and an external camera (not shown), in particular in the form of a stereo camera, which detects and evaluates the pose of the marker on the surgical microscope. This enables additional tracking of the pose of the surgical microscope relative to the patient.

This surgical 3D marker element 1 has a sterilizable surface 2 in which a two-dimensional optical pattern 4 is permanently incorporated, in particular is printed on or engraved, for spatial pose recognition of a medical product 100. Specifically, the optical pattern 4 is configured in the form of a matrix 6 with M=10 rows and N=5 columns with corresponding pattern elements 8 (10×5=50) as pattern fields 8.

The optical pattern 4 has more than four pattern elements 10 with the fifty, of which two pattern elements 10 are configured differently in order to provide an optical reference for pose detection of the 3D marker element 1 by the optical tracking system 500. In particular, the optical pattern is at least partially positioned around the suction pipe 110 so that the camera 504 can detect at least a part of the optical pattern 4 from each side or in each pose of the suction pipe. The pose may still be determined on the basis of this part of the optical pattern 4.

The tracking system 500 is specifically adapted to optically detect the 3D marker element 1 with the optical pattern 4 and to determine a pose on the basis of the detected optical pattern 4 and to include it in its navigation system or its navigation function, and to visually output an up-to-date pose to the surgeon via a display device 506 in conjunction with preoperative image data such as CT image and MRI image. In this way, the suction pipe 110 may be displayed in the microscope image and the exact pose of the suction pipe 110 may also be displayed in a section of MRI data in a further view in order to ensure reliable and good navigation. In particular, landmarks may also be set and the suction pipe 110 may also be used as a pointer. The suction pipe 110 does not have to be elaborately adapted, but in this configuration it is sufficient to use a commercially available suction pipe, to engrave the optical pattern directly into the surface 2 of the distal portion using a laser, and then to optically detect it. The 3D marker element thus provides an efficient and cost-effective way of adapting already approved medical products and making them ready for pose recognition and upgrading them. In this way, it may be raised to the next functional level without the need for complex additional components, which would usually result in lengthy and cost-intensive approval. In particular, however, the optical pattern may be used to perform very precise pose recognition, since it may be arranged in particular in the immediate vicinity of an operating point of the medical product and may even extend over a wide area, so that pose recognition may still be performed even if an instrument tip or a tip of the suction pipe 110 is obscured and pose recognition may even be performed reliably from different directions (even if part of the optical pattern 4 is obscured).

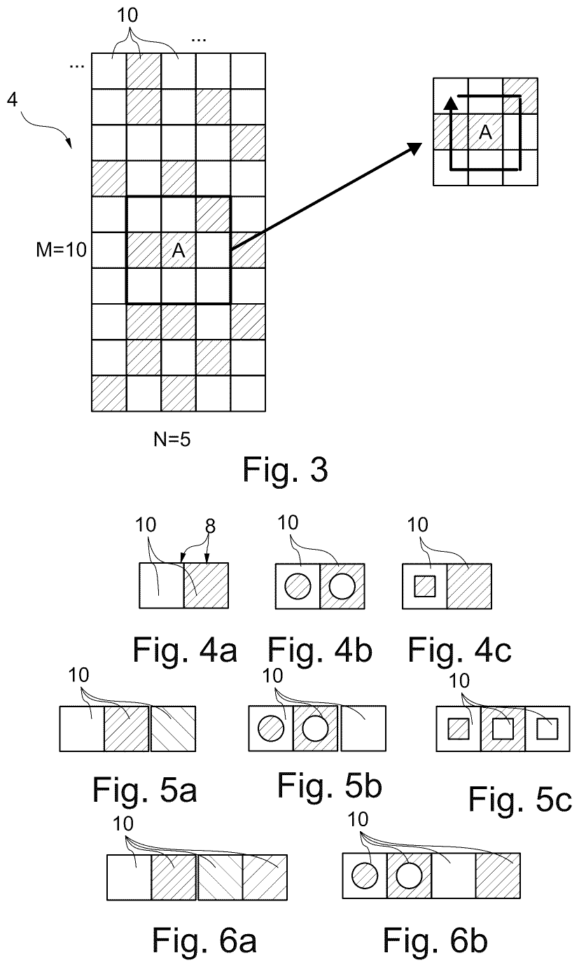

FIG. 2 shows an example of a matrix with M by N cells or pattern fields in which pattern elements can be inserted (see FIG. 3). In FIG. 2, the matrix is rectangular with square pattern fields 8, which all have the same outline contour, i.e. are configured in the same way.

Moving on to FIG. 3, these pattern fields 8 are equipped with pattern elements 10, whereby in FIG. 3 binary pattern elements 10 are configured with a first light (here white) pattern element 10 and a second dark (here black) pattern element 10 (the hatching is intended to represent a dark, in particular black, color). The result is an optical pattern 4 of first and second pattern elements 10, which have been arranged according to a definition rule and, so to speak, optically encode the information or make it detectable (by a computer).

In FIG. 3, a cell group of 3×3 pattern elements is shown outlined in the optical pattern 4 (left part) in a central area for an overview and is shown individually in a right part of FIG. 3 for an explanation. A central pattern element 10 (dark) is surrounded by 8 other neighboring pattern elements. These nine pattern elements 10 form a pattern word. In particular, the tracking system 500 may determine on the basis of the pattern word that binary pattern elements 10 are used.

FIGS. 4a to 4c each show different (pairs) of binary pattern elements 10. FIG. 4a shows a light (e.g. white) pattern field 8 as pattern element and a dark (e.g. black-hatching is intended to represent dark or black) pattern field as pattern element 10. In FIG. 4b, a filled circle is also provided in the middle of the pattern field (may also be described as a dot), in the respectively other color. Alternatively, as shown in FIG. 4c, a rectangle in the form of a square may be used instead of a circle.

FIGS. 5a to 5c show further embodiments of (triples) ternary pattern elements 10. These are similar to the binary ones in FIGS. 4a to 4c, with the difference that a further pattern field 8 is added with a further, different pattern element 10.

FIGS. 6a and 6b show alternative ternary (quadruple) on quaternary pattern elements 10. All these group pairs, triples and quadruples may each be stored as group entries in a pattern dictionary, so that the tracking system 500 knows the respective pairs, triples, or quadruples and the tracking system may then determine the various pattern elements 10 and then at least a pose on the basis of this pattern dictionary.

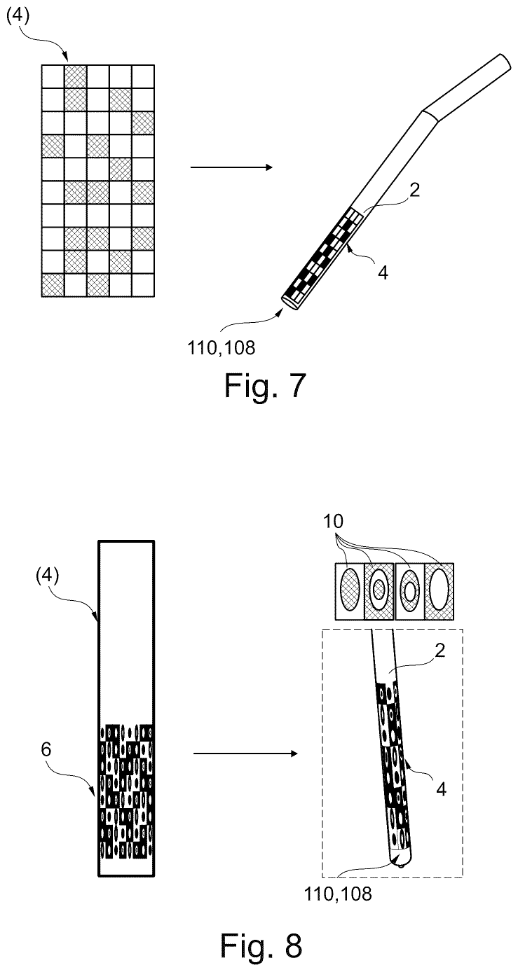

FIG. 7 shows an example of a template of an optical pattern (left part) as well as a corresponding insertion of the optical pattern 4 into the surface 2 of the suction pipe 110. The pattern is rolled or wrapped, so to speak, over the cylindrical surface of the suction pipe and forms the 3D marker element 1 integrated into the medical device. In particular, the optical pattern 4 may have further information on the apparatus/instrument type (coded) and may be combined with data associated with the apparatus or instrument.

FIG. 8 shows a further embodiment of a template of a 3D marking element with the optical pattern (left part), which is then applied to a distal portion 108 of the suction pipe 110. In this embodiment, a quadruple is used as a quaternary pattern element 10, which has ellipses and nested ellipses. With just two ‘colors’ or contrasts, four different elements may be created using the different designs of the pattern elements 10 and provided accordingly for at least one pose recognition on an instrument or apparatus. Above the suction pipe 110 on the right-hand side of FIG. 8, the group of pattern elements 10 is shown once again for illustration purposes (as a character set of pattern elements, so to speak).

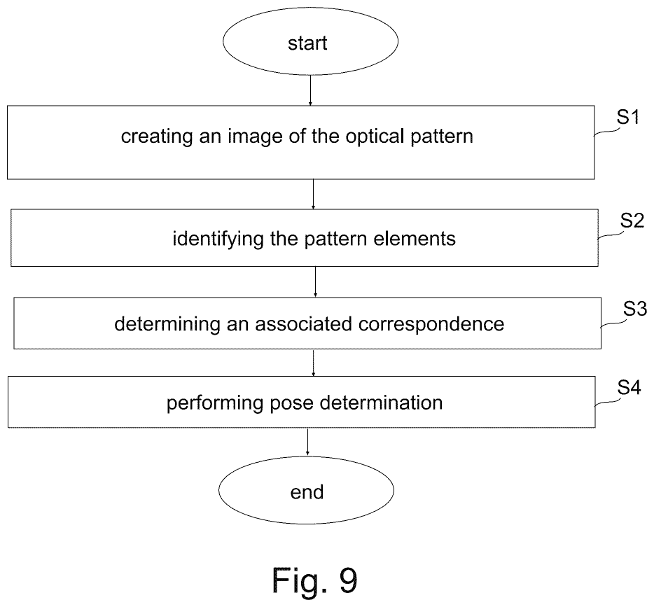

FIG. 9 shows in a flow chart a pose recognition method for a pose recognition of a 3D marker element 1, with an optical pattern 4, in particular for the tracking system 500 as described above. This method has the following steps.

In a first step S1, a 2D image is created by the camera 504 of a tracking unit 502 or a 3D image is created by at least two cameras of a tracking unit 502 and each visible pattern element 10 of the optical pattern 4 is detected.

In a subsequent step S2, each pattern element 10 is identified by a control unit 510 on the basis of a dictionary with different pattern elements 10, in particular binary pattern elements (10).

Then, in step S3, a correspondence between a 2D position in the 2D image and a position of the template of the 3D marker element 1 with the optical pattern 4 or a correspondence between a 3D position in the 3D image and a position of the template of the 3D marker element 1 with the optical pattern 4 is determined.

Finally, in step S4, an n-point perspective position determination is performed on the 2D image for a pose determination or a 3D-to-3D point determination for a pose determination of the optical pattern 4.

The position as well as the orientation of the 3D marker element 1 is then determined with the optical pattern 4 and may be integrated into a navigation accordingly.

In particular, precise and unambiguous pose recognition can therefore be carried out using only one (conventional) 2D image (i.e. a two-dimensional image) via the defined optical pattern 4.

### LIST OF REFERENCE SIGNS

1 3D marker element
2 surface
4 optical pattern
6 matrix
8 pattern field/cell field
10 pattern element/
100 medical product
102 instrument
104 medical apparatus
106 instrument tip
108 distal portion
110 suction pipe
500 tracking system
502 tracking unit
504 camera
506 display device
508 storage unit
510 control unit
S1 step creating an image
S2 step identifying pattern elements
S3 step determining correspondence
S4 step performing pose determination

1-28. (canceled)

29. A surgical tracking system for a surgical intervention on a patient, the surgical tracking system comprising:
a medical product having a 3D marker element for spatial pose recognition of the medical product; and
a tracking unit with at least one camera and a display device,
the 3D marker element comprising a sterilizable surface and an optical pattern that is two-dimensional,
the optical pattern being permanently incorporated in the sterilizable surface,
the optical pattern comprising a matrix with M rows and N columns,
the optical pattern further comprising at least four pattern elements,
at least two of the at least four pattern elements configured differently in order to provide an optical reference for pose detection of the 3D marker element via an optical tracking system,
the at least one camera adapted to optically detect the 3D marker element with the optical pattern and determine a pose based on the optical pattern and visually output the pose by the display device,
the 3D marker element being integrated directly into the medical product, and
the optical pattern being printed on or incorporated into the sterilizable surface of the medical product.

30. The surgical tracking system according to claim 29, further comprising a storage unit and a control unit, wherein:
a dictionary of pattern elements is stored in the storage unit,
the control unit is adapted to read out the optical pattern based on the dictionary and determine the pose, and
a part of the optical pattern is sufficient to determine the pose.

31. The surgical tracking system according to claim 30, wherein a data set associated with the optical pattern is stored in the storage unit, wherein the data set comprises a transformation from the pose of the optical pattern to an operating point of the medical product, in order to detect a position of the operating point as well as an orientation of a distal portion of the medical product, and the control unit is adapted to determine the operating point as well as orientation of the distal portion based on a pose recognition of the optical pattern as well as the transformation, and to output them visually in a view by the display device.

32. The surgical tracking system according to claim 29, wherein any portion of the optical pattern is usable for pose determination.

33. The surgical tracking system according to claim 29, wherein the optical pattern is usable for pose determination purely optically and without using information encoded in the optical pattern.

34. The surgical tracking system according to claim 33, wherein the optical pattern is not a barcode and/or is not a QR code.

35. The surgical tracking system according to claim 29, wherein:
the optical pattern has at least three rows by three columns,
the at least four pattern elements comprise nine pattern elements,
the nine pattern elements comprise a central pattern element and eight other pattern elements each adjacent to the central pattern element, and
the nine pattern elements together form a pattern word and are uniquely identifiable.

36. The surgical tracking system according to claim 29, wherein:
the sterilizable surface is curved, and
a first portion of the optical pattern is configured to be offset and/or inclined with respect to a second portion of the optical pattern such that the first portion and the second portion are not visible simultaneously.

37. The surgical tracking system according to claim 29, wherein the at least four pattern elements are distinguished by color and/or center of a circle, and/or corners and/or contours.

38. The surgical tracking system according to claim 29, wherein the medical product is a surgical instrument with a distal instrument tip and the 3D marker element is arranged in an area less than 50% of a total length of a distal instrument tip to provide a spatial pose recognition of the distal instrument tip, wherein the surgical instrument is a surgical scalpel and has the 3D marker element on a flat surface of a cutting edge of the surgical scalpel.

39. The surgical tracking system according to claim 29, wherein the medical product has a cylindrical contour with a corresponding cylindrical surface on a distal portion, and the 3D marker element is arranged partially circumferentially, wherein the medical product is a medical suction device with a distal suction pipe and the optical pattern is printed on or engraved into a surface of the distal suction pipe.

40. The surgical tracking system according to claim 29, wherein:
the medical product is configured as a medical instrument with a conical geometry of a distal portion, and
an optical template pattern comprising a rectangular matrix is mapped adapted to the conical geometry via a linear transformation matrix and is present as an optical pattern adapted on or in the sterilizable surface.

41. The surgical tracking system according to claim 29, wherein the medical product is a surgical instrument.

42. The surgical tracking system according to claim 29, wherein the medical product is a medical suction device.

43. A method for pose recognition of a 3D marker element on a surgical tracking system according to claim 29, the method comprising the steps of:
creating a 2D image by the at least one camera of the tracking unit and detecting each visible pattern element of the optical pattern;
identifying each pattern element by a control unit based on a dictionary with different pattern elements;
determining a correspondence between a 2D position in the 2D image and a position of a template of the 3D marker element with the optical pattern; and
performing an n-point perspective position determination in the 2D image for a pose determination of the optical pattern.

44. A method for pose recognition of a 3D marker element on a surgical tracking system according to claim 29, the method comprising the steps of:
creating a 3D image by at least two cameras of the tracking unit and detecting each visible pattern element of the optical pattern;
identifying each pattern element by a control unit based on a dictionary with different pattern elements;
determining a correspondence between a 3D position in the 3D image and a position of a template of the 3D marker element with the optical pattern; and
performing a 3D-to-3D-point determination for a pose determination of the optical pattern.
